(19) United States
(12) Patent Application Publication
(10) Pub. No.: US 2024/0115872 A1
(43) Pub. Date: Apr. 11, 2024

(54) ELECTROMAGNETIC IMPLANT FOR TREATMENT OF SOLID CANCERS

(71) Applicant: Ohio State Innovation Foundation, Columbus, OH (US)

(72) Inventors: Vishwanath Subramaniam, Toronto (CA); Joseph West, Richwood, OH (US)

(21) Appl. No.: 18/270,286

(22) PCT Filed: Dec. 30, 2021
(86) PCT No.: PCT/US2021/065634

§ 371 Date: Jun. 29, 2023

Related U.S. Application Data
(60) Provisional application No. 63/132,066, filed on Dec. 30, 2020.

(51) Int. Cl. A61N 2/02; A61N 2/00

(52) U.S. Cl. CPC A61N 2/02; A61N 2/004

(57) ABSTRACT

An implant device (105) such as an antennae or coil of wire (107) is implanted into or around a group of cancerous cells such as a tumor (120). A magnetic field generator (205) such as a magnet or electromagnet is placed (310) close to the implant device such that the implant device is within a magnetic field (215) generated (315) by the magnetic field generator. The magnetic field is changed (330), by rotating or moving the magnetic field generator, such that an electric field is induced within the implant device and the group of cancerous cells. The electric field has been shown to reduce the size or number of the cancerous cells, to reduce the growth rate of the cancerous cells, and to halt or reduce metastasis. The strength and frequency of the electric field can be changed based on the type of cancerous cells or based on how the cancerous cells respond to the implant device.

### CROSS-REFERENCE TO RELATED APPLICATIONS

This application claims priority to U.S. provisional application No. 63/132,066, filed on Dec. 30, 2020, and titled “ELECTROMAGNETIC IMPLANT FOR TREATMENT OF SOLID CANCERS,” the disclosure of which is hereby incorporated by reference in its entirety.

### BACKGROUND

Non-contact induced electric fields, generated by time-dependent magnetic fields produced by current-carrying coils driven at 100 kHz, have been shown to selectively hinder the migration of highly metastatic breast cancer cells in vitro. Similar effects have been observed in vitro with other cells such as keratinocytes and murine wound macrophages. These induced electric fields have been shown, in a direction dependent manner, to either slow down or completely arrest some of the migrating cancer cells in microchannels mimicking the topography of preexisting paths formed by vessels, extracellular matrix fibers, and white matter tracts in the brain that guide migrating cancer cells in vivo.

However, while induced electric fields show promise in the treatment of certain cancers, there is currently no way to selectively target cancer cells using induced electric fields without also subjecting non-cancerous cells to the induced electric fields.

It is with respect to these and other considerations that the various aspects and embodiments of the present disclosure are presented.

### SUMMARY

In an embodiment, an implant device such as an antenna or coil of wire is implanted into or around a group of cancerous cells such as a tumor. A magnetic field generator, such as a magnet or an electromagnet, is placed sufficiently close to the implant device such that the implant device is within a magnetic field generated by the magnetic field generator. The magnetic field is varied in time either by changing the current in a coil in the generator in a time-dependent manner, or by rotating or moving the magnet, such that an electric field is induced within the implant device and the group of cancerous cells. The induced electric field has been shown to reduce the number of the cancerous cells that migrate and with the right dosing is expected to reduce the growth rate of the cancerous cells. The strength and frequency of the induced electric field can be changed based on the type of cancerous cells or based on how the cancerous cells respond to the treatment. The strength and frequency of the electric field can be changed by changing one or more of the strengths of the magnetic field, the rate of change of the magnetic field, spatial variation of the magnetic field, the number of windings in the coil of wire, and by using a capacitor in the implant device.

In an embodiment, a method for treatment of solid cancers is provided. The method includes: placing an implant device near or within a tumor; generating a magnetic field around the implant device; and changing the magnetic field to induce an electric field in the implant device and the tumor.

Embodiments may include some or all of the following features. The tumor may include cancer cells. The tumor may include one or more of breast cancer cells, prostate cancer, pancreas cancer cells, liver cancer cells, colon cancer cells, or stomach cancer cells. the implant device may include a biocompatible casing and a coil comprising a number of windings. The implant device may include a capacitor. The method may further include adjusting one or both of the number of windings and a size of the capacitor to increase or to decrease a size of the electric field or the frequency of the electric field. The method may further include adjusting a strength of the magnetic field or a rate of change of the magnetic field to increase or to decrease a size of the electric field. Generating the magnetic field may include using a magnet. Changing the magnetic field may include rotating the magnet. Generating the magnetic field may include driving a current that varies with time through an external coil. The induced electric field in the tumor results in one or more of a reduction of a size of the tumor, a reduction in a rate of growth of the tumor, a reduction of a number of migrating cells of the tumor, or a reduction of the speed with which cells migrate. The method may further include: determining a change in a voltage or a current in the implant device; and determining a change in a size of the tumor based on the determined change in the voltage or current.

In an embodiment, an implant device for treatment of solid cancers is provided. The implant device includes: a coil comprising a number of windings; a capacitor; and a biocompatible housing that encases the coil and capacitor, and wherein the implant device is configured to be implanted near or within a tumor, and to generate an electric field in the tumor when exposed to a changing magnetic field.

Embodiments may include some or all of the following features. The tumor may include cancer cells. The tumor may include one or more of breast cancer cells, prostate cancer, pancreatic cancer cells, liver cancer cells, colon cancer cells, or stomach cancer cells. Other types of cancer cells may be supported. At least one of the number of windings or a size of the capacitor may be adjustable to increase or to decrease a size of the electric field and a frequency of the electric field. The magnetic field is generated using a magnet or electromagnetic device.

In an embodiment, a system is provided. The system includes: an implant device adapted to be placed near or within a tumor; and a magnetic field generating device, wherein the magnetic field generating device is adapted to: generate a magnetic field around the implant device; and change the magnetic field to induce an electric field in the implant device and the tumor.

Embodiments may include some or all of the following features. The implant device may include a biocompatible casing and a coil comprising a number of windings. The electric field in the tumor may result in at least one of a reduction of a size of the tumor, a reduction in a rate of growth of the tumor, or a reduction of a number of migrating cells of the tumor. The implant device may include a capacitor.

Treating cancer using an implant device as described herein provides many advantages over the prior art methods of treating cancer. First, by using the implant device, the induced electric field can be localized in the tumor or its microenvironment, thereby protecting non-cancerous cells as well as having any systemic effects as in the case of chemotherapy. Second, because the induced electric field is amplified by the implant device, a weaker overall electric field can be used to further protect the non-cancerous cells. Third, an externally applied induced electric field is dependent on the frequencies supported by the external device applying the electric field. When using the implant device described herein, the frequencies of the induced electric field can be adjusted by adjusting properties of the implant device such as the number of windings in the coil or the size and number of capacitors. Fourth, because of relatively small size of the implant device and the magnetic field generator used to invoke the electric field in the implant device, subjects (e.g., patients) may be provided with a portable device which allows them to receive treatment in the comfort of their homes and offices or woven into the fabric of their clothing. Fifth, the implant device may be used instead of, or in conjunction with, existing cancer treatments such as chemotherapy, immunotherapy, and radiation. Where the implant device is used in conjunction with such treatments, it may reduce the time or amount of the treatments which may help subjects avoid or mitigate certain side effects associated with the treatments.

This summary is provided to introduce a selection of concepts in a simplified form that are further described below in the detailed description. This summary is not intended to identify key features or essential features of the claimed subject matter, nor is it intended to be used to limit the scope of the claimed subject matter

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1A and FIG. 1B are illustrations of exemplary implant devices;

FIG. 2 is an illustration of an exemplary magnetic field generating device;

FIG. 3 is an illustration of an exemplary method for inducing an electric field in an implant device;

FIG. 4 is an illustration of an exemplary wearable magnetic field generating device;

FIG. 5 is an illustration of an exemplary wearable magnetic field generating device woven into an article of clothing;

FIG. 6 is an illustration of lactate dehydrogenase and succinate dehydrogenase activities in MDA-MB-231 cells and MCF10A cells with and without induced electric fields and epidermal growth factor; and

FIG. 7 is an illustration of relative baseline oxygen consumption and relative baseline glycolysis of MDA-MB-231, MCF10CA1a, and MCF10A cells after 12 hours of induced electric field treatments with or without 25 ng/mL epidermal growth factor supplement.

### DETAILED DESCRIPTION

This description provides examples not intended to limit the scope of the appended claims. The figures generally indicate the features of the examples, where it is understood and appreciated that like reference numerals are used to refer to like elements. Reference in the specification to “one embodiment” or “an embodiment” or “an example embodiment” means that a particular feature, structure, or characteristic described is included in at least one embodiment described herein and does not imply that the feature, structure, or characteristic is present in all embodiments described herein.

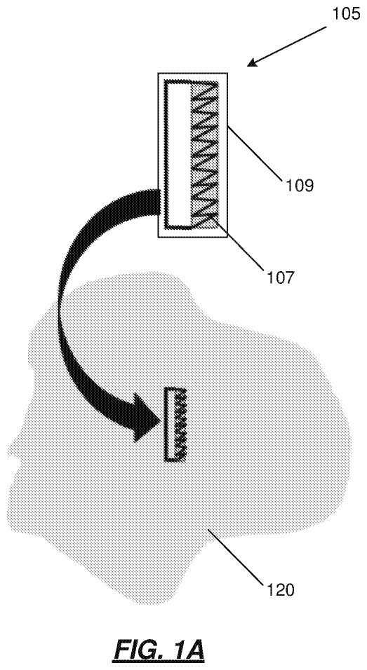

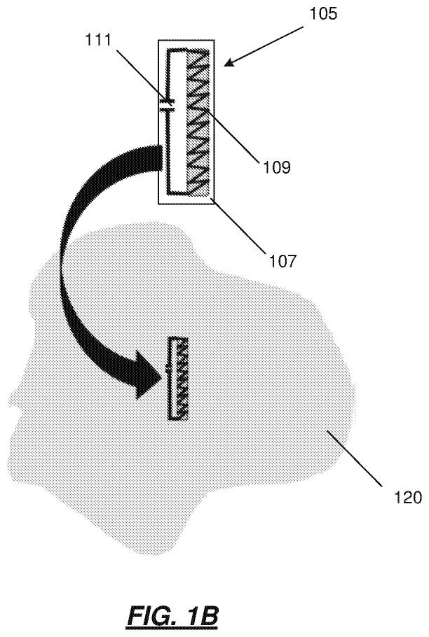

FIG. 1A is an illustration of an example implant device 105. As shown, the implant device 105 may include a coil 107 placed within a biocompatible casing 109. The implant device 105 may be sized such that it can be placed within or around a tumor 120 of a human or an animal subject. The tumor 120 may include a group of cancerous cells including, but not limited to, breast cancer, prostate cancer, pancreas cancer, liver cancer, colon cancer, and stomach cancer. The coil 107 may be designed such that the coil 107 creates an induced electric field when subject to a changing magnetic field. The induced electric field may reduce the size of number of cancerous cells in the tumor 120, may reduce a rate of growth of the cancerous cells in the tumor 120, or may halt or reduce metastasis.

The biocompatible casing 109 may be constructed from a variety of biomechanical materials including, but not limited to, alumina, bio glass, hydroxyapatite, silicone, polyethylene glycol (PEG) and polyvinylchloride. Other materials may be used. Preferably the casing is non-shielded, non-conducting, and non-magnetic such that it does not interfere with any electric fields generated by the implant device 105.

In some embodiments, the ends of the coil 107 may be connected to each other to form a closed path for current flow in the implant device 105. Alternatively, the ends of the coil 107 may be left open or disconnected. Leaving the ends of the coil 107 open may result in no current flow in the coil 107 but may result in a charge separation between the ends of the coil 107 in the implant device 105.

FIG. 1B is an illustration of another example implant device 105. Similar to the implant device of FIG. 1A, the implant device 105 may also include a coil 107 within a biocompatible housing 109. In addition, the implant device 105 of FIG. 1B further includes a capacitor 111 connected to the coil 107 in series. Depending on the embodiment, multiple capacitors 111 may be used. The use of the capacitor 111 in the implant device 105 may result in a frequency for the invoked electric field as the capacitor 111 is repeatedly charged and discharged.

The implant device 105 may be sized so that it can be implanted or placed in or near a tumor 120 non-invasively. As example size may be less than 1 cm×1 cm×1 cm. Other sizes may be supported.

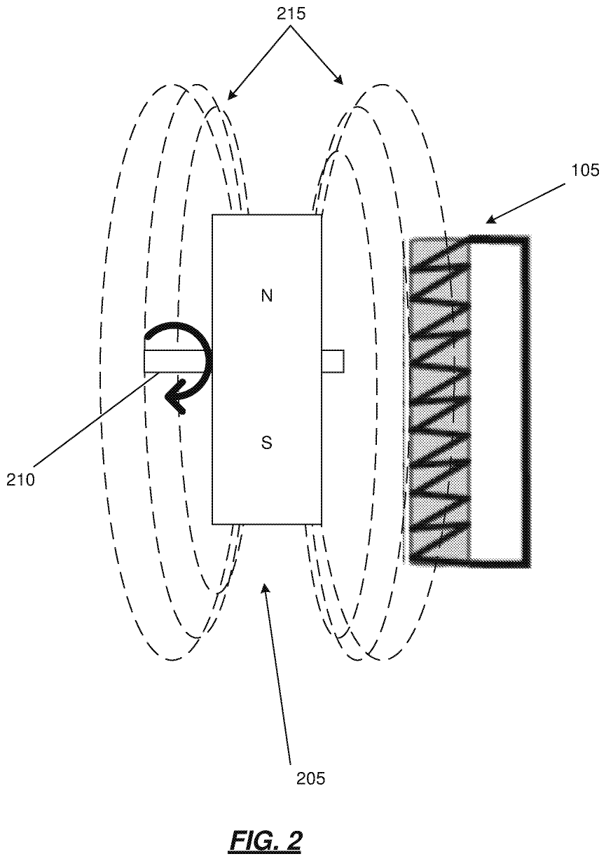

FIG. 2 is an illustration of an exemplary magnetic field generating device 205 that is configured to generate a magnetic field 215. As shown, the magnetic field generating device 205 is a magnet; however, other types of magnetic field generating devices may be supported such as an electromagnet (e.g., a wire coil).

When the magnetic field generating device 205 rotates around an axis 210, the change in the magnetic field 215 induces an electric field in the coil 107 of the implant device 105 (with or without the capacitor 111). The strength of the induced electric field is described by Faraday's law and may depend on a variety of factors such as the strength of the magnetic field 215, the rate of change (e.g., due to AC current flow or an externally rotating magnet) of the magnetic field 215, and a number of windings in the coil 107 (i.e., inductance) of the implant device 105.

In order to treat cancerous cells in a human or an animal subject, the implant device 105 may be used in conjunction with the magnetic field generating device 205 to generate a controlled induced electric field at or around the cancerous cells. Initially, the characteristics of the implant device 105 may selected based on the type of cancer cells being treated. These selected characteristics may affect both the strength of the induced electric field that is generated and the frequency of the induced electric field. Example characteristics that may be selected include the size and the thickness of the coil 107, the number of windings of the coil 107, and the size and the number of capacitors 111 (if any). In some embodiments, the selected coil 107 may be a 47 mH inductor connected in series with a 1346 pF capacitor, resulting in a resonant frequency of ˜20 kHz. These parameters may be varied to accommodate resonance frequencies less than about 1 MHz.

The implant device 105 may be placed at or around the location of the cancerous cells of a tumor 120. Depending on the type of cancerous cells, the implant device 105 may be surgically placed within or near the tumor 120 of the human or the animal subject by a medical provider. The implant device 105 may be implanted by the medical provider such that when the electric field is induced in the implant device 105, the cancerous cells of the tumor 120 are within the electric field. Depending on the embodiment, where the tumor 120 is external or accessible without using surgery, the implant device 105 may be affixed to the subject on or near the tumor 120.

After the implant device 105 is placed on or in the subject, the magnetic field generating device 205 may be brought near to the implant device 105 by the medical provider such that the implant device 105 is sufficiently within the magnetic field 215 generated by the device 205. This may ensure that the desired electric field is induced in the implant device 105 at the tumor 120 due to changes in the magnetic field generated by the device 205. In some embodiments, the magnetic field generating device 205 may be placed approximately 1-2 cm from the implant device 105.

In some embodiments, the magnetic field generating device 205 may include a power source that drives an AC current through an external coil, or two moving (rotating or translating) magnets each producing a 1.3 T magnetic induction (B) field 215. Other strength magnets may be used. The magnets may be mounted on a rotor whose rotation may be controlled to achieve a desired rate of change for the resulting magnetic field 215. Other methods for rotating the magnets or device 205 may be used.

After the magnetic field generating device 205 is brought near the implant device 105, the medical provider may turn on the device 205 such that the magnetic field 215 is generated and the rotation of the device 205 results in a change to the magnetic field 215 at a frequency that is based on the rotation speed of the device 205. In one example, the device 205 may rotate at approximately 2200 rpm corresponding to a frequency of approximately 2200 rpm. This corresponds to a frequency of approximately 73 Hz and a time varying B field of ˜73 Hz. Other frequencies may be used.

The change in the magnetic field 215 results in an induced electric field in the implant device 105. The strength of the induced electric field can be adjusted either by changing the strength of the externally applied magnetic field, or the frequency of the changing magnetic field or both. Alternatively, if the subject is moving in space, the magnetic field from the generator could be constant in time but non-homogeneous (i.e., varying) in space. Because the implant device 105 is near or within the set of cancerous cells that make up the tumor 120, an induced electric field is also received in the cancerous cells of the tumor 120. This induced electric field may reduce the size or number of cancerous cells of the tumor 120, may reduce a growth rate of the tumor 120, or may halt or reduce metastasis. In particular, the induced electric field may drive the tumor 120 to eventual necrosis by promoting apoptosis of just the cancerous cells of the tumor 120 or turn the tumor fibrotic and incapable of metastasis.

Depending on the embodiment, a subject may receive regular daily treatment from the implant device 105 and the magnetic field generating device 205 for some predetermined amount of time (e.g., one week, one month, one year, etc.). Each treatment may have a duration that ranges anywhere from one hour to twenty four hours, for example. After a subject (e.g., patient) completes a course of treatment, the associated medical provider may use standard diagnostic techniques (e.g., CT scans, PET scans, ultrasound, biopsy, etc.) to determine whether the treatment has been effective (e.g., has the tumor reduced in size or has the tumor's growth rate been reduced). Depending on the diagnosis, the medical provider may adjust one or more characteristics of the implant device 105 to either increase or decrease one or both of a strength of the induced electric field or the frequency of the induced electric field.

In some embodiments, the magnetic field generating device 205 may be a wearable device, and the subject with the implant device 105 may use the wearable magnetic field generating device 205 to perform regular treatments on themselves while at home or at work. In such embodiments, the subject may wear the magnetic field generating device 205 for long periods of time and possibly even while sleeping. The subject may check in regularly with a medical provider to determine if the treatments are effective or if any changes to the duration or strength of treatments may be needed.

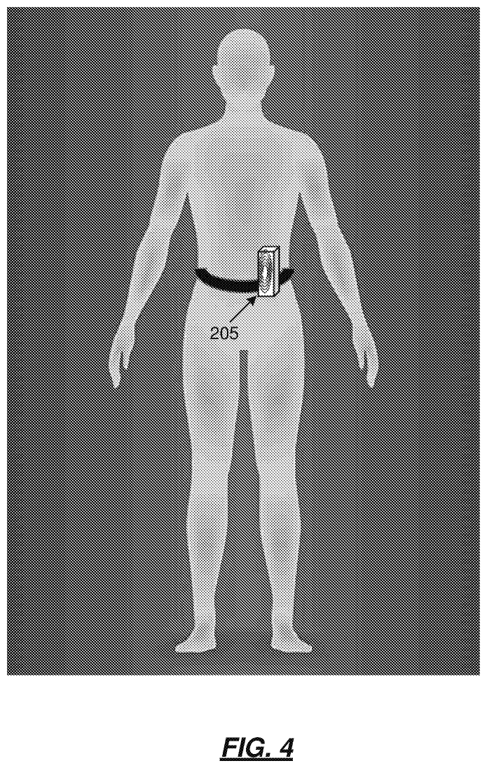

For example, FIG. 4 is an illustration of an exemplary wearable magnetic field generating device 205. As shown, the device 205 includes a belt that allows the device 205 to be strapped to the subject.

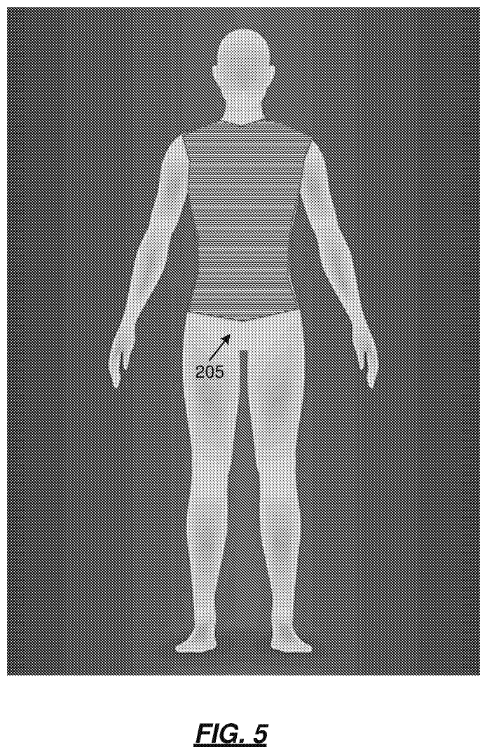

In some embodiments, the wearable magnetic field device 205 may be incorporated into an article of clothing. For example, wires or other conductive materials may be woven into the fabric of an article of clothing. When an electric current is provided to the conductive materials, a magnetic field maybe generated around and within the article of clothing. FIG. 5 is an illustration of a device 205 incorporated into an article of clothing such as a shirt. The black stripes in the shirt represent the conductive materials or wires woven into the shirt.

The induced electric field treatments described herein may have the most impact on inoperable tumors, including pancreatic and prostate cancers. Though the morbidity in prostate cancers is not as bad as for other cancers, the fact that surgical intervention can lead to changes in quality of life makes it a potentially good candidate for induced electric field intervention.

For breast cancer, the notion of a surgical margin may not be relevant because by nature it is a conservative surgery whereby margins are deliberately reduced to as safe levels as possible in order to preserve breast tissue. The induced electric field treatments described herein may be helpful for not only in hindering metastasis but also ensuring the success of partial lumpectomies.

For prostate cancer, the implant device 105 could be used in place of existing treatments like radiation beads. The treatment could be for days, weeks, months, or even longer. Metastasis can be hindered as long as an induced electric field is present and therefore the subject may want to continue treatment on a permanent basis.

In some embodiments, the implant device 105 may also be used to monitor the size and/or progression of a tumor 120. In particular, characteristics of the implant device 105 such as voltage and current may be monitored while the magnetic field 215 is received. Any changes in the monitored voltage or current may also indicate a corresponding change in the size of the tumor 120. Accordingly, the effectiveness of the implant device 105 may be determined in part based on the change in the characteristics.

In some embodiments, current can be induced in a small coil 107 of an implant device 105 whose size can be varied according to the size of a primary tumor 120, by a time varying magnetic field 215 applied remotely (1-2 cm) from the coil 107. The external field could be generated by a moving magnet (rotating or translating) or by another EM coil through which an alternating current is driven by an external power source. Alternatively, the EM energy could be beamed by an antenna focused on the smaller coil 107. The ends of the smaller coil 107 may be joined to form a closed circuit or the circuit may be closed with another electrical element (resistor, capacitor, and/or inductor) of which a capacitor 111 could be a critical element to cause a time varying current to flow at a specific frequency of choice (20 kHz in the example shown). The current in this coil 107 produces an induced electric field inside the tumor 120, but in a locally magnified form so as to produce an induced electric field inside the tumor 120 that is larger in magnitude compared to the induced electric field induced by the externally applied B field. Such a coil 107 excited in this manner can locally amplify and concentrate induced electric fields within the tumor 120 so as to either shrink it, maintain its size and prevent it from growing further, or prevent it from metastasizing and spreading to other parts of the body.

The implant device 105 described herein can be used with animal and human patients. With respect to an animal patient such as a mouse, standard protocol immortalized metastatic breast cancer cells (e.g. MDA-MB-231) can be injected into the #4 mammary fat pad of an FVB (named for its susceptibility to Friend leukemia Virus B) mouse. This model is an excellent model for metastasis as it reproduces progression of metastatic disease in humans, with the breast cancer metastasizing to the lungs upon injection of the MDA-MB-231 cells in the mammary fact pads of the mice. The same model can be used for the invention disclosed here as follows. An implant device 105 including an EM coil 107 with closed ends or connect to a capacitor 111 and encased in a biocompatible material (e.g., polyethylene glycol or PEG, or polyethylenterephthalate or PET) and of a small enough size that is compatible for insertion into the No. 4 mammary fat bad of an FVB mouse may be implanted into the tumor 120 by minimally invasive surgery. A current is made to flow in this inserted implant device 105, thereby generating a magnetic field 215, which in turn induces an induced electric field inside the tumor 120. This may be accomplished by one of several ways, the easiest of which is an external magnetic field 215 produced by a current carrying wire surrounding the implant device 105. This current in the primary external coil generates a time-varying magnetic field 215, which in turn induces a current in the implanted coil 107 of the implant device 105. The current made to flow in this manner in the implanted coil 107 then generates its own magnetic field 215 which in turn produces an induced electric field in the tumor 120, hindering or preventing metastasis, while either maintaining or reducing the volume of the primary tumor 120.

With respect to human patients, the current in the implant device 105 (and the induced electric field in the tumor 120) can be introduced in a non-contact manner by having an external magnetic field generating device 205 (either woven into a wearable fabric or contained within a portable pack as in the case of chemo pumps) which also houses a power source to drive a current through the external coil, or other objects (neck bands, blankets, mattresses, etc.) comprising one or more coils. As in the case of the mouse patient, the current in the primary external coil generates a time-varying magnetic field 215, which in turn induces a current in the implanted coil 107 of the implant device 105. The current made to flow in this manner in the implanted coil 107 then generates its own magnetic field 215 which in turn produces an induced electric field in in the tumor 120, hindering or preventing metastasis, while either maintaining or reducing the volume of the primary tumor 120. An advantage of this two-coil system as opposed to the single coil, non-contact induced electric field therapy is that the induced electric field in the case of the implant device 120 is concentrated in the primary tumor 120 or portion of the organism/body, versus subjecting the entire organism or body to the induced electric field.

Alternatively, even with the whole body subjected to a time-varying magnetic via an enclosure or chamber (analogous to hyperbaric chambers used in the treatment of chronic wounds), the implanted coil 107 of the implant device 105 can still produce a stronger and concentrated induced electric field compared to single coil, non-contact technologies. The device and method described here is intended to hinder or eliminate metastasis of a primary tumor 120 without necessarily killing primary tumor cells but may also either arrest the growth of the primary tumor 120 or even shrink it. The ability to treat metastatic disease without necessarily killing the primary tumor 120 or shrinking it, differentiates both non-contact induced electric field therapy and induced electric field implant therapy.

It is expected that cellular changes associated with curtailing cancer cell metastasis by an induced electric field implant device may in certain circumstances be similar to some of the changes by a non-contact (i.e., non-implant) induced electric field device. These include a reduction in the recruitment of tumor associated macrophages (TAMs) in the tumor microenvironment (TME); a reduction in the number of cancer stem-like cells in the TME; a decrease of epithelial-to-mesenchymal (EMT) transition markers in the primary tumor; and an increase in the expression of E-cadherin in the primary tumor, a critical indicator of an intact epithelium and cell-cell adhesion.

In addition to halting metastasis, the implant device 105 is capable of reporting (as a sensor would) about the status of the primary tumor 120. Electrical characteristics of tumors 120 are known to be different than surrounding tissue, and with induced electric field implant therapy, the electrical characteristics of the tumor are expected to change as the therapy progresses. Just as the implant device 105 in the primary tumor generates an induced electric field in the tumor 120, the changing electrical properties of the tumor 120 in turn alter the electrical signal within the implanted coil 107 and can cause changes in the electrical signatures of either an external coil or a tertiary device (comprising another coil specifically designed for this purpose), which can then be detected. In this manner, the status of the induced electric field implant therapy may be monitored in real time and adjusted if and when necessary.

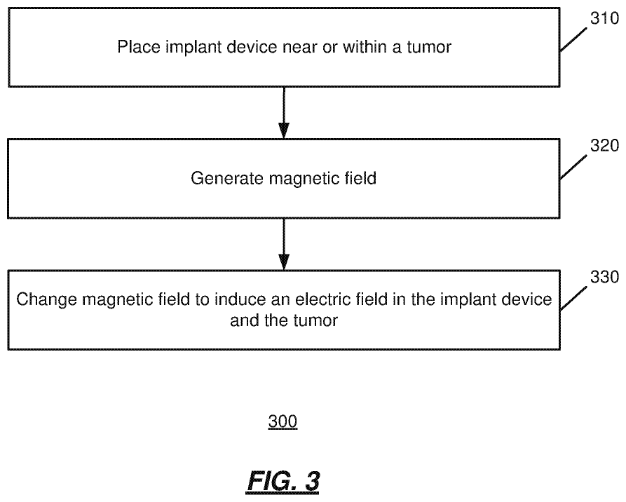

FIG. 3 is an illustration of an example method 300 for inducing an electric field as treatment for cancer in a human or an animal subject. The method 300 may be implemented using an implant device 105 and a magnetic field generating device 205.

At 310, an implant device is placed near or within a tumor. The implant device 105 may be placed near or within a tumor 120 on the body of a human or an animal subject by a medical provider. The implant device 105 may include a coil 107 within a biomechanical housing 109. The implant device 105 may be placed such that an induced electric field in the implant device 105 passes through or is received by one or more cancerous cells of the tumor 120. The implant device 105 may be surgically implanted within the tumor 120 or placed near the tumor 120 using an adhesive or other affixing means.

At 320, a magnetic field is generated around the implant device. The magnetic field 215 may be generated around the implant device 105 by a magnetic field generating device 205 such as a magnet. The magnetic field generating device 205 may be placed sufficiently close to the implant device 105 such that the magnetic field 215 generated by the magnetic field generating device 205 passes through the implant device 105. Depending on the embodiment, the magnetic field generating device 205 may be a wearable device so that the subject can receive treatment from the implant device 105 and device 205 over extended periods of time including when the subject is at home, at work, or sleeping.

At 330, the magnetic field is changed to induce an electric field in the implant device and the tumor. The magnetic field 215 may be changed by the magnetic field generating device 205 rotating the magnet associated with the magnetic field 215 about an axis. The magnetic field may also be generated and changed by driving a current through an external coil. The changing of the magnetic field 215 may result in an induced electric field in the implant device 105 and the tumor 120. The strength and the frequency of the resulting induced electric field may be controlled by adjusting one or more of the number of windings in the coil 107 of the implant device 105, the size of the capacitor 111 (if any) used in the implant device 105, the strength of the magnetic field 215 generated by the magnetic field generating device 205, and distance between the implant device 105 and the magnetic field generating device 205. The electric field may be induced for a time sufficient to reduce a size of the tumor 120, to reduce a rate of growth of the tumor 120, or to halt or reduce metastasis. The time, frequency, and strength of the induced electric field may be dependent on the type of cancer cells that make up the tumor 120.

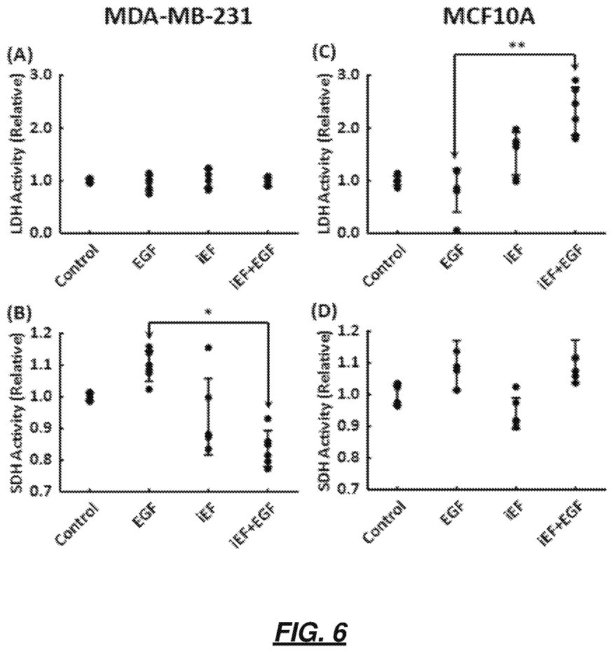

FIG. 6 is an illustration of lactate dehydrogenase (“LDH”) (A) and succinate dehydrogenase (“SDH”) (B) activities in MDA-MB-231 cells with and without induced electric fields and epidermal growth factor (“EGF”). FIG. 6 further illustrates LDH (C) and SDH (D) activities in MCF10A cells with and without induced electric fields and EGF. Results are shown for N=3 separate experiments (3 separate 6-well plates on separate days). Values have been normalized to the control condition. Black circles represent the calculated activities from each individual well across 3 experiments. Error bars represent one standard deviation above and below the mean. *p<0.05.

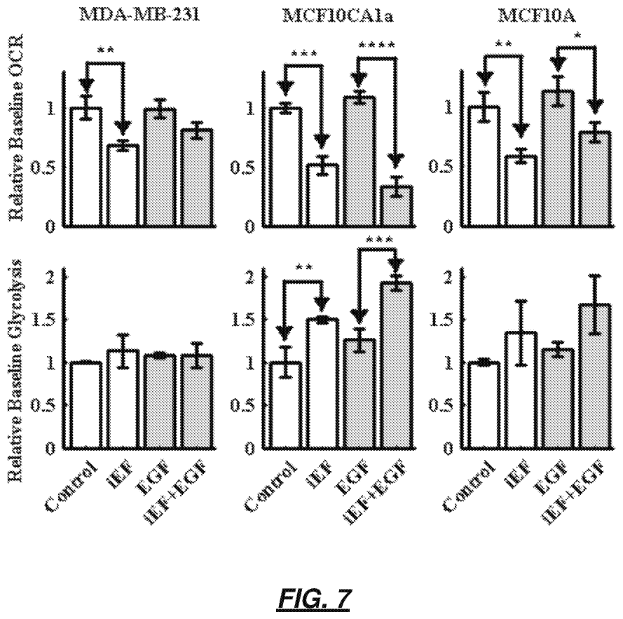

FIG. 7 is an illustration of relative baseline oxygen consumption (“OCR”) and relative baseline glycolysis of MDA-MB-231, MCF10CA1a, and MCF10A cells after 12 hours of induced electric field treatments with or without 25 ng/mL EGF supplement. Shaded bars represent conditions with EGF. *p<0.05, **p<0.01, ***p<0.001, ****p<0.0001.

As used herein, the singular form “a,” “an,” and “the” include plural references unless the context clearly dictates otherwise.

As used herein, the terms “can,” “may,” “optionally,” “can optionally,” and “may optionally” are used interchangeably and are meant to include cases in which the condition occurs as well as cases in which the condition does not occur.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

While the present invention is disclosed in several forms, it will be apparent to those skilled in the art that many modifications can be made therein without departing from the spirit and scope of the present invention and its equivalents. Therefore, other modifications or embodiments as may be suggested by the teachings herein are particularly reserved.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method for treatment of solid cancers, the method comprising:
placing an implant device near or within a tumor;
generating a magnetic field around the implant device; and
changing the magnetic field to induce an electric field in the implant device and the tumor.

2. The method of claim 1, wherein the tumor comprises cancer cells.

3. The method of claim 2, wherein the tumor comprises one or more of breast cancer cells, prostate cancer, pancreas cancer cells, liver cancer cells, colon cancer cells, or stomach cancer cells.

4. The method of claim 1, wherein the implant device comprises a biocompatible casing and a coil comprising a number of windings.

5. The method of claim 4, wherein the implant device further comprises a capacitor.

6. The method of claim 5, further comprising adjusting one or both of the number of windings and a size of the capacitor to increase or to decrease a size of the electric field or a frequency of the electric field.

7. The method of claim 1, further comprising adjusting a strength of the magnetic field or a rate of change of the magnetic field or a spatial variation of the magnetic field to increase or to decrease a size of the electric field.

8. The method of claim 1, wherein generating the magnetic field comprises generating the magnetic field using a magnet.

9. The method of claim 8, wherein changing the magnetic field comprises rotating the magnet.

10. The method of claim 1, wherein the induced electric field in the tumor results in one or more of a reduction of a size of the tumor, a reduction in a rate of growth of the tumor, or a reduction of a number of migrating cells of the tumor.

11. The method of claim 1, further comprising:
determining a change in a voltage or a current in the implant device; and
determining a change in a size of the tumor based on the determined change in the voltage or current.

12. An implant device for treatment of solid cancers, the implant device comprising:
a coil comprising a number of windings;
a capacitor; and
a biocompatible housing that encases the coil and capacitor; and
wherein the implant device is configured to be implanted near or within a tumor, and to generate an electric field in the tumor when exposed to a changing magnetic field.

13. The implant device of claim 12, wherein the tumor comprises cancer cells.

14. The implant device of claim 12, wherein the tumor comprises one or more of breast cancer cells, prostate cancer, pancreas cancer cells, liver cancer cells, colon cancer cells, or stomach cancer cells.

15. The implant device of claim 12, wherein at least one of the number of windings or a size of the capacitor are adjustable to increase or to decrease a size of the electric field and a frequency of the electric field.

16. The implant device of claim 12, wherein the magnetic field is generated using a magnet.

17. A system comprising:
an implant device adapted to be placed near or within a tumor; and
a magnetic field generating device, wherein the magnetic field generating device is adapted to:
generate a magnetic field around the implant device; and
change the magnetic field to induce an electric field in the implant device and the tumor.

18. The system of claim 17, wherein the implant device comprises a biocompatible casing and a coil comprising a number of windings.

19. The system of claim 17, wherein the electric field in the tumor results in at least one of a reduction of a size of the tumor, a reduction in a rate of growth of the tumor, or a reduction of a number of migrating cells of the tumor.

20. The system of claim 17, wherein the implant device comprises a capacitor.
